(19) United States
(12) Patent Application Publication
(10) Pub. No.: US 2026/0047859 A1
(43) Pub. Date: Feb. 19, 2026

(54) HOLLOW NEEDLE, KIT, AND METHOD FOR CREATING VOIDS IN TISSUE

(71) Applicant: Asahi Intecc Co., Ltd., Seto-shi (JP)

(72) Inventor: Hirobumi Watanabe, Irvine, CA (US)

(21) Appl. No.: 19/300,325

(22) Filed: Aug. 14, 2025

Related U.S. Application Data
(60) Provisional application No. 63/683,857, filed on Aug. 16, 2024.

(51) Int. Cl. A61B 17/3205; A61B 17/00; A61B 17/34; A61B 90/00

(52) U.S. Cl. CPC A61B 17/32053; A61B 17/3421; A61B 2017/00561; A61B 2017/00743; A61B 2017/3454; A61B 2090/064; A61B 2217/005

(57) ABSTRACT

Embodiments of the present disclosure relate to a hollow needle configured to form a void in a membrane. An example hollow needle includes a tube body that extends along a tube axis between a first end and a second end, wherein the first end defines a tip of the tube body; and a hollow portion surrounded by the tube body, wherein the hollow portion comprises an opening in the tip. The tube body may comprise: a sharp portion provided at the tip; and a suction void connected to the hollow portion that may be fluidly connected to a suction device, wherein the hollow portion is configured to cause a negative pressure applied to the suction void to be applied through the opening. The application of the negative pressure through the opening may pull the membrane against the tip to cause the sharp portion to pierce the membrane.

### CROSS-REFERENCE TO RELATED APPLICATIONS

This claims priority to U.S. Provisional App. No. 63/683,857, filed Aug. 16, 2024, the content of which is hereby incorporated by reference in its entirety.

### TECHNICAL FIELD

This application generally relates to hollow needles, kits, and methods for making a void in a membrane.

### BACKGROUND

Surgery on a brain may be performed for various medical purposes. The brain is surrounded by a thin membrane called a dura mater which is about 0.3 mm thick. To access the brain surface, the dura mater is breached. Incisions to the dura mater may be necessary to measure electrophysiological brain activity, reduce abnormal intracranial water pressure, exclude bleeding, resect brain tissue (e.g., cancer, arteriovenous malformations, traumatic brain energy, and/or the like), sample brain tissue, and/or the like. For example, an electrode array may be placed on the brain surface for procedures to diagnose epilepsy. The placement of this electrode requires making a void in a skull and the dura mater. The conventional surgical procedure involves exposing the brain tissue by drilling a void into the skull and cutting a straight or cross-shaped incision through the dura mater using a scalpel. Another approach includes piercing through the skull and dura mater via a needle to access subdural tissues. However, there exists a need to reduce the size of such incisions to achieve a more minimally invasive surgery. Further, there exists a need to reduce the likelihood of damaging subdural tissues during formation of a void through the dura.

Minimally invasive surgical procedures typically require more specialized devices as compared to conventional techniques. In a minimally invasive brain surgery, access to the organ is spatially limited, which may render conventional drill and scalpel techniques unsuitable for safely exposing brain tissue. For example, a conventional open skull surgery may require a skull flap as large as 10 cm to sufficiently expose brain tissue, whereas a minimally invasive surgery may seek to inspect the brain tissue via a fiberscope passed through a void as small as 1 mm. Accordingly, new tools may be required to safely and accurately access brain tissue on a scale that is orders of magnitude smaller than conventional techniques.

### BRIEF SUMMARY

Embodiments of the present disclosure relate to hollow needles, hollow needle kits, and methods for using the same. An example hollow needle of the present disclosure includes a tube body that extends along a tube axis between a first end and a second end, wherein: the first end defines a tip of the tube body; the tip comprises a sharp portion; and the second end defines a suction void configured to receive a negative pressurization from a suction device; a hollow portion surrounded by the tube body, wherein the hollow portion extends through and defines an opening of the tip, wherein: the hollow portion is configured to apply the negative pressure through the opening to suction a membrane to the tip; and the sharp portion is configured to cut the membrane via rotation of the tube body.

In some embodiments, the tube body comprises multiple sharp portions that are in an annular arrangement at the tip. In some embodiments, the sharp portion comprises a protrusion comprising: a sharp tip configured to pierce the membrane; and a side edge configured to cut the membrane via rotation of the tube body. In some embodiments, the side edge is arranged substantially parallel to the tube axis. In some embodiments, the tube body further comprises a flat surface at the tip that is arranged to be substantially perpendicular to the tube axis; and the sharp portion protrudes from the flat surface. In some embodiments, the sharp portion protrudes from the flat surface by 0.2 mm to 4.0 mm. In some embodiments, the sharp portion protrudes from the flat surface by at least 0.3 mm.

In some embodiments, the hollow portion is closed in the second end; and the suction void is provided at a circumferential portion of the tube body between the tip and the second end. In some embodiments, the tube body further comprises an engagement shaft configured to connect to a rotating tool. In some embodiments, the engagement shaft is provided at the second end. In some embodiments, the engagement shaft comprises a solid, flat portion configured to contact and halt advancement of the tip into the membrane. In some embodiments, a diameter of the tube body is 0.5 mm to 0.7 mm. In some embodiments, the membrane is a dura mater that surrounds a brain.

In various embodiments, a kit is provided that includes one or more hollow needles as described herein and shown in the drawings. In some embodiments, the kit further comprises a needle holder comprising: a holder body comprising a connector configured to be fluidly connected to the suction device; and a bearing configured to rotatably support the tube body, wherein the needle holder is configured to receive the suction void. In some embodiments, the bearing is further configured to provide an airtight seal between the holder body and the tube body.

In some embodiments, the tube body further comprises an engagement shaft configured to connect to a rotating tool, wherein the engagement shaft is provided at the second end of the tube body; the holder body comprises a needle holding portion and a connector portion; the tube body extends into the needle holding portion such that the tip and the second end extend from the needle holding portion; and the connector portion comprises the connector. In some embodiments, the kit further comprises the suction device that may be fluidly connected to the connector. In some embodiments, the kit further comprises a sensor configured to measure the negative pressure; and an indicator configured to report a degree of the negative pressure measured by the sensor. In some embodiments, the tube body further comprises an engagement shaft; and the kit further comprises a rotating tool configured to hold the engagement shaft.

In various embodiments, a method for making a void in a membrane is provided. In some embodiments, the method includes applying the negative pressure at the tip by the suction device; and advancing the tip toward the membrane, wherein the negative pressure pulls a portion of the membrane to the tip such that the sharp portion pierces the portion of the membrane. In some embodiments, the method further comprises halting advancement of the tip toward the membrane in response to detecting an increase in the negative pressure. In some embodiments, the method further comprises rotating the tube body to cause the sharp portion to cut a void into the portion of the membrane. In some embodiments, the method further comprises halting rotation of the tube body in response to detecting a decrease in the negative pressure. In some embodiments, the method further comprises suctioning into the hollow portion a cut fragment of the portion of the membrane.

### BRIEF DESCRIPTION OF THE FIGURES

Having thus described the embodiments of the disclosure in general terms, reference now will be made to the accompanying drawings, which are not necessarily drawn to scale, and wherein:

FIG. 1 shows a perspective view of an example kit in accordance with some embodiments of the present disclosure;

FIG. 2 shows a perspective view of an example hollow needle in accordance with some embodiments of the present disclosure;

FIG. 3A shows a left partial perspective view of an example hollow needle in accordance with some embodiments of the present disclosure;

FIG. 3B shows a right partial perspective view of an example hollow needle in accordance with some embodiments of the present disclosure;

FIG. 3C shows a top view of an example hollow needle in accordance with some embodiments of the present disclosure;

FIG. 3D shows a partial side view of an example hollow needle in accordance with some embodiments of the present disclosure;

FIG. 4 shows a perspective view of an example needle holder cut in half along an axis in accordance with some embodiments of the present disclosure;

FIG. 5 shows a perspective view of an example bearing cut in half along an axis in accordance with some embodiments of the present disclosure;

FIG. 6 shows a perspective view of an example rotating tool with some embodiments of the present disclosure;

FIG. 7 shows a perspective view of an example kit in accordance with some embodiments of the present disclosure;

FIG. 8 shows a side view of an example hollow needle in accordance with some embodiments of the present disclosure;

FIG. 9 shows a partial perspective view of an example hollow needle in accordance with some embodiments of the present disclosure;

FIGS. 10A-10E shows schematic diagrams of an example hollow needle in accordance with some embodiments of the present disclosure.

### DETAILED DESCRIPTION

Some embodiments of the present invention will now be described more fully hereinafter with reference to the accompanying drawings, in which some, but not all, embodiments of the invention are shown. Like reference numerals refer to like elements throughout. Indeed, various embodiments of the invention may be embodied in many different forms and should not be construed as limited to the embodiments set forth herein. Rather, these embodiments are provided so that this disclosure will satisfy applicable legal requirements.

As used herein, the term “or” is used in both the alternative and conjunctive sense, unless otherwise indicated. The term “along,” and similarly utilized terms, means near or on, but not necessarily requiring directly on an edge or other referenced location. The terms “approximately,” “generally,” and “substantially” refer to within manufacturing and/or engineering design tolerances for the corresponding materials and/or elements unless otherwise indicated. Thus, use of any such aforementioned terms, or similarly interchangeable terms, should not be taken to limit the spirit and scope of embodiments of the present invention.

As used herein, reference is made to a hollow needle for making a void in a dura mater. The present disclosure, however, contemplates that the hollow needles of the present disclosure may be equally applicable to other applications for making a small void in a membrane. For example, the hollow needle may be used in other procedures, such as in epicardial access via subxiphoid puncture, and/or the like.

### Overview

In general, various embodiments of the present disclosure provide improved designs for forming voids in dura mater via a hollow needle. Specifically, the hollow needles described herein may be utilized to form small diameter voids for supporting minimally invasive brain surgeries. The various hollow needles described herein and shown in the figures may demonstrate efficient and safe designs as compared to conventional tools for cutting a dura mater. In doing so, the present needle may overcome challenges associated with making voids of reduced diameter as compared to incisions used in existing approaches. It will be understood and appreciated that such context is provided by way of example and uses of the hollow needle in additional contexts, such as with other medical procedures, are contemplated and within the scope of the invention.

As described above, existing tools face challenges in minimizing a void in the durra mater. For example, it may be difficult for surgeons to accurately and precisely cut the dura in small areas using a scalpel. Further, use of a scalpel or other convention cutting instrument may increase a risk of damaging brain tissues.

To solve these issues and others, example implementations of embodiments of the present application may provide a hollow needle comprising a tube body that comprises a sharp portion at a tip and a hollow portion that comprises an opening in the tip. The simple structure of the hollow needle may assist in operator's simple procedures compared to the conventional use of a scalpel. The hollow needle may be configured to be rotated to make a round void in the dura mater. The sharp portion of the hollow needle may allow for efficient cut in the right size for the designated device (e.g., a sensor guidewire) to go through. The round void made by the hollow needle may minimize the size of the incision of the dura mater. The round void may reduce the surface area of the dura mater that is removed to expose subdural tissues. For example, in comparison to conventional approaches that utilize a plurality of line cuts to remove a portion of the dura mater, the hollow needle described herein may remove a portion of the dura mater via a single, circular cut. In some embodiments, the circular cut of the hollow needle reduces a likelihood of tear propagation along the dura mater. For example, conventional approaches may produce stress concentrations along the line cuts and, in doing so, increase a risk of tear propagation along the cut paths.

In some embodiments, the hollow needle comprises a flat surface at the tip. The structure of the tip of the hollow needle may reduce the risk of damaging brain tissues. In some embodiments, the flat surface is configured to contact the dura mater and oppose further advancement of the sharp portion through the dura mater, which would otherwise risk penetration into subdural tissues. For example, when the flat surface contacts the dura mater, the penetration of the sharp portion may be halted. In some embodiments, the height of the sharp portion protruding from the flat surface is 0.2 mm to 4.0 mm. For example, the height of the sharp portion may be 0.3 mm. As another example, the height of the sharp portion may be 2.0 mm. In some embodiments, the height of the sharp portion may within a threshold range less than the thickness of the target membrane to be cut. For example, the height of the sharp portion may be 0.1 mm to 2.0 mm less than the thickness of a target segment of dura mater. In some embodiments, the appropriate height prevents the sharp portion from protruding behind the dura mater when the sharp portion goes into the dura mater. For example, a less than 1:1 ratio of the height to the thickness of the target membrane may reduce a likelihood of the sharp portion extending to subdural tissues.

In some embodiments, the hollow needle comprises a hollow portion comprising an opening. In some embodiments, a negative pressure is applied to the hollow portion. In such contexts, the negative pressure may be further applied to the dura mater. The application of negative pressure may cause the dura mater to displace from subdural brain tissues. In this manner, the risk of piercing subdural brain tissues during creation of the void may be reduced. For example, by pulling the dura mater away from the brain tissues, the hollow needle may more safely cut the dura mater without damaging the brain tissues. In some embodiments, the negative pressure causes the tip of the hollow needle to pierce the membrane. In some embodiments, while the negative pressure holds the membrane against the tip, the hollow needle is rotated to cause the tip to cut through the membrane in a circular manner. In this manner, a circular segment of material may be cut from the membrane, thereby forming a void through the membrane.

In various embodiments, the negative pressure may be constantly applied during the procedures to establish a void in the dura mater. In some embodiments, the constant application of the negative pressure enables the operator to realize the timings to stop moving the hollow needle toward the dura mater after the contact and rotating the hollow needle after creating the void. In some embodiments, the negative pressure may be detected by a sensor and a degree of the negative pressure is indicated by an indicator. The steps of the procedures may be confirmed by monitoring the change of negative pressure. The indication may assist the operator in confirming milestones for transitioning between operations of the procedure (e.g., advancement to dura mater, rotation of the hollow needle, retreat from dura mater, and/or the like). The negative pressure may increase upon the hollow needle contacting the dura mater, which may indicate to the operator that the hollow needle has reached the target site of void formation and advancement may be halted. While the dura mater is suctioned against the tip, the hollow needle may be rotated to cut the dura mater and, in doing, create a void. When the void is created in the dura mater, the negative pressure may decrease. The decrease in pressure may indicate to the operator that the void has been formed in the dura mater.

In this manner, the hollow needle described hereafter improves efficiency and safety of making a small diameter void in the dura or other membrane structures. The structure of the tip of the hollow needle may improve surgical accuracy and reduce a likelihood of damaging subdural tissues. Further, the application of the negative pressure applied to the tip may also improve the operation and reduce the risk of the damage. In doing so, the hollow needle may achieve adequate procedure for making a void in a membrane such that the operator may make a small void easily, effectively, and safely.

### Example Kit Including Hollow Needle

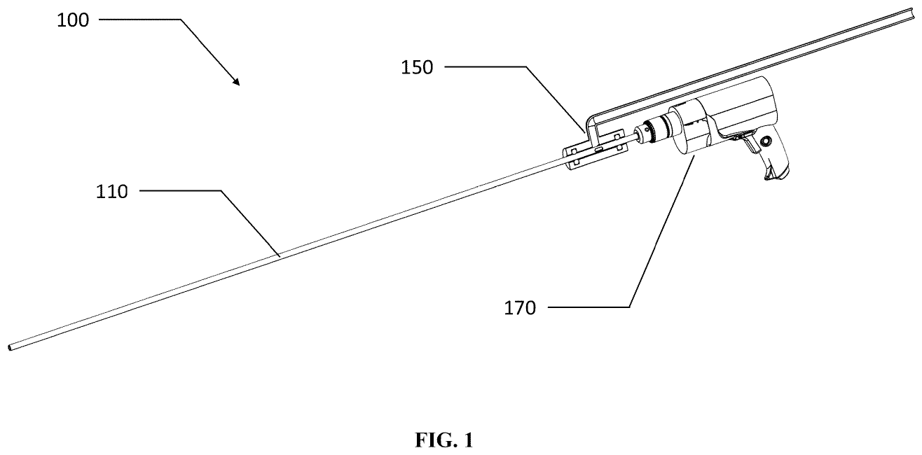

With reference to FIG. 1, shown is a perspective view of an example kit 100 for making a void in a membrane such as a dura. In some embodiments, the kit 100 includes a hollow needle 110, a needle holder 150, and a rotating tool 170. In various embodiments, the hollow needle 110 is configured to cut a membrane, such as a dura mater. For example, a tip of the hollow needle 110 may comprise an annular arrangement of sharpened edges such that a membrane held against the tip may be cut via rotation of the hollow needle 110. In some embodiments, the hollow needle 110 is rotatably supported by the needle holder 150. In some embodiments, the rotating tool 170 is connected to the hollow needle 110 such that torque from the rotating tool 170 may be transmitted to the hollow needle 110. Additionally, or alternatively, the hollow needle 110 may be gripped and manipulated by an operator. In some embodiments, the kit 100 includes a suction device (e.g., a pump, and/or the like) that may be connected to the needle holder 150. In various embodiments, the suction device is configured to generate a negative pressure within the needle holder 150 and hollow needle 110. The hollow needle 110 may be positioned adjacent to a membrane, and the negative pressure may suction a portion of the membrane to the tip of the hollow needle 110. The hollow needle 110 may be rotated to create a circular incision defining a void through the membrane.

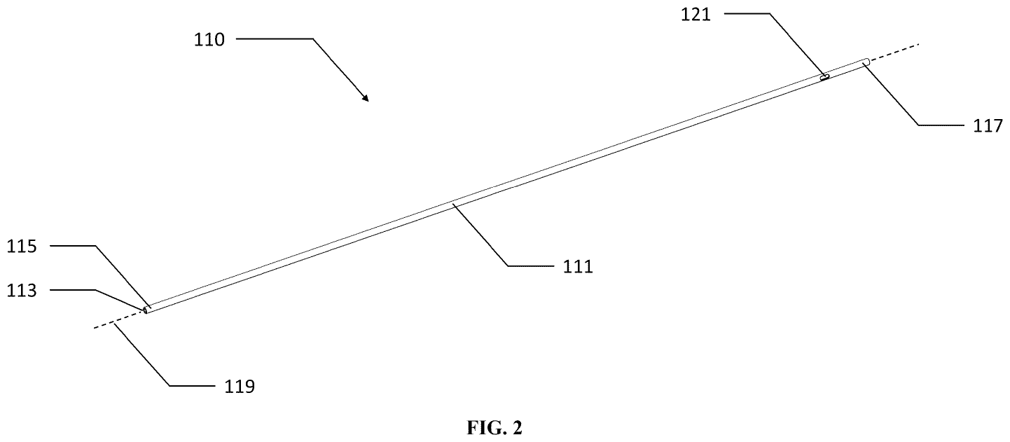

FIG. 2 shows a perspective view of an example hollow needle 110. As shown, the hollow needle 110 may comprise a tube body 111 comprising a hollow portion 113. The hollow portion 113 may be surrounded by the tube body 111. In some embodiments, the tube body 111 includes a first end defining a tip 115 and a second end 117 opposite the tip 115. The tube body 111 may extend between the tip 115 and the second end 117. The terms, the tip 115 and the second end 117 may refer to designated edges of the tube body and areas of the tube body 111 proximate to the designated edges. In some embodiments, the tube body 111 includes a circular cross-section. In some embodiments, the tube body 111 has a tube axis 119 along which the tube body 111 extends longitudinally. For example, the tube axis 119 may be a center of a circular cross-section of the tube body 111. In some embodiments, the hollow needle 110 is rotatable about the tube axis 119.

In some embodiments, the tip 115 of the tube body 111 is configured to pierce and cut a membrane. The hollow portion 113 may comprise an opening in the tip 115. The hollow portion 113 may be closed in the second end 117. In various embodiments, the tube body 111 comprises an engagement shaft that may be held by a rotating tool 170. The engagement shaft may include a solid and flat portion. For example, the engagement shaft may be provided at the second end 117.

In some embodiments, the tube body 111 comprises a suction void 121 that is connected to the hollow portion 113. The suction void 121 may be provided at a circumferential portion of the tube body 111 between the tip 115 and the second end 117. The suction void 121 may be provided at a rear portion of the tube body 111, for example at proximate to the second end 117. In some embodiments, the suction void 121 may be fluidly connected to a suction device such that the vacuum created by the suction device transmits to the hollow portion 113. In various embodiments, the hollow portion 113 is configured to cause a negative pressure applied to the suction void 121 to be applied through the opening in the tip 115. In various embodiments, the negative pressure is configured to pull the dura mater away from the brain tissues and cause the sharp portion 131 to penetrate the dura mater. In some embodiments, the negative pressure is 0.1 atmospheres (atm) to 1.0 atm. For example, an electromechanical pump, syringe pump, and/or the like may be connected to the hollow needle 110 and activated to generate a negative pressure of 1.0 atm within the tube body 111 and at the tip 115.

In some embodiments, the tube body 111 includes stainless steel, tungsten rhenium, titanium, tungsten carbide, and/or the like. In various embodiments, the diameter of the tube body 111 is 0.5 mm to 0.7 mm. For example, the diameter of the tube may be 0.6 mm.

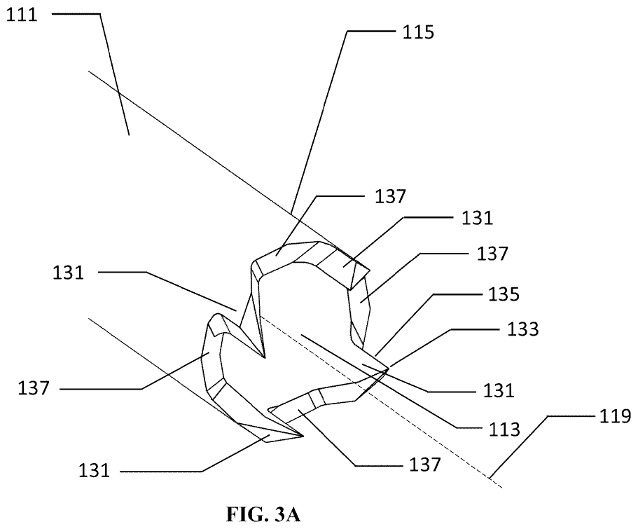

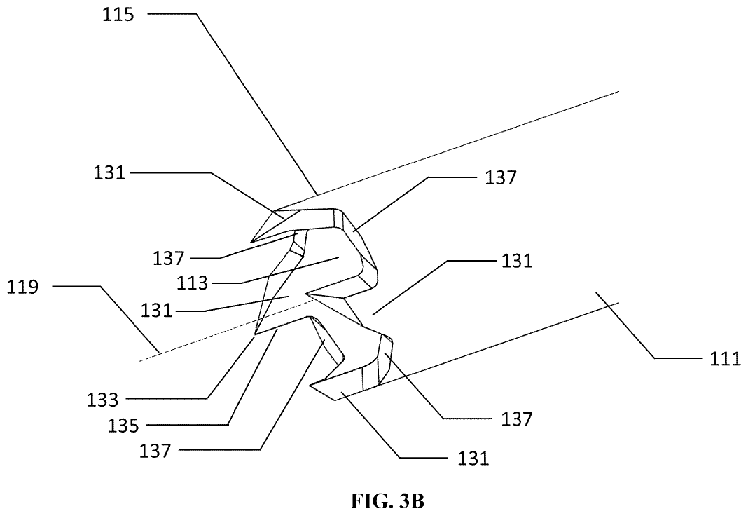

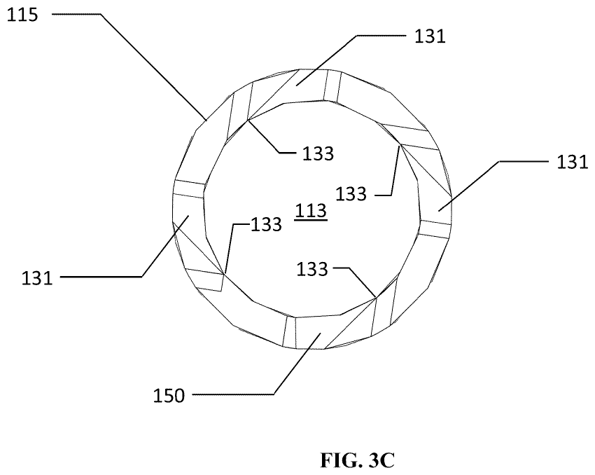

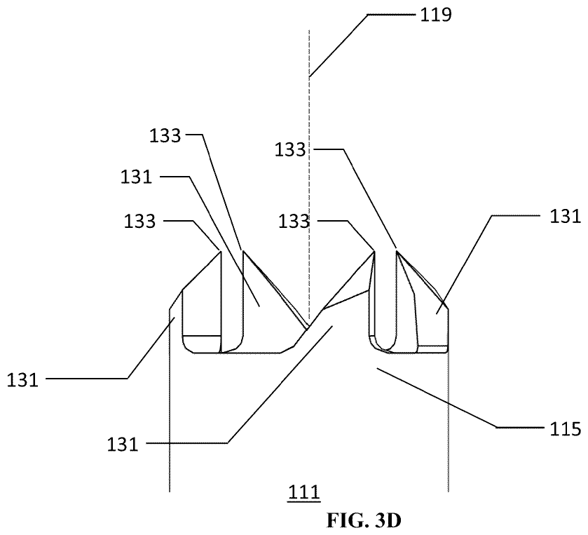

FIG. 3A and FIG. 3B show, respectively, left partial perspective and right partial perspective views of an example hollow needle 110. In various embodiments, the tip 115 of the tube body 111 includes a plurality of sharp portions 131 to cut the membrane. For example, the tube body has four sharp portions. The sharp portions 131 may comprise blade structures that constitute a circular serration provided at the tip 115. In some embodiments, the sharp portions 131 are positioned in an annular arrangement at the tip 115. In some embodiments, the sharp portions 131 comprise an identical structure relative to one another. In some embodiments, the identical structures of the sharp portions 131 more evenly distributes stress and, in doing so, reduces a risk of destructive shearing, torsion, and/or the like. Alternatively, in some embodiments, one or more sharp portions 131 may demonstrate varying heights, edge angles, and/or the like.

In some embodiments, the sharp portion 131 comprises a protrusion that has a sharp tip 133 at the front and a side edge 135 that extends toward the sharp tip 133. The side edge 135 may comprise a sharp portion that tapers off gradually in a circumferential direction. For example, the side edge 135 may extend substantially parallel to the tube axis 119. In various embodiments, when cutting a membrane, the sharp tip 133 initially penetrates into a membrane, and the side edge 135 further cuts the membrane as the hollow needle 110 is rotated. In some embodiments, a respective sharp portion 131 may comprise a single side edge 135 at one side in the circumferential direction such that the sharp portion 131 may cut the membrane only when the hollow needle 110 is rotated in a predetermined direction (e.g., only clockwise or only anticlockwise). Alternatively, in some embodiments, a respective sharp portion 131 may comprise two opposed side edges 135 (e.g., at both sides in the circumferential direction) such that the sharp portion 131 may cut the membrane when the hollow needle 110 is rotated in a first or a second direction (e.g., clockwise or anticlockwise).

In some embodiments, the tube body 111 comprises flat surfaces 137 at the tip 115. The flat surfaces 137 may be arranged to be substantially perpendicular to the tube axis 119. In some embodiments, the sharp portion 131 protrudes from the flat surface 137. The flat surfaces 137 may be arranged between the side edges 135. In various embodiments, when the sharp portion 131 bites into the dura mater, the flat surface contacts the dura mater and prevents the sharp portion 131 from penetrating into brain tissues. In some embodiments, the height of the sharp portion 131 protruding from the flat surface is 0.2 mm to 4.0 mm. For example, the height of the sharp portion 131 may be 0.3 mm, which may be the thickness of the dura mater. The height of the sharp portion may within a threshold range of the thickness of the membrane to be cut. For example, the height of the sharp portion may be equal to the membrane thickness. Alternatively, the height of the sharp portion may be within a threshold range less than the membrane thickness. The appropriate height of the sharp portion 131 may prevent the sharp portion from protruding behind the dura mater when the sharp portion 131 goes into the dura mater.

In some embodiments, the negative pressure applied through the opening in the tip 115 pulls the membrane so that the sharp portion 131 bites into the membrane. After that the side edge 135 may cut the membrane and make a round void in the membrane by the rotation of the hollow needle 110.

FIG. 3C shows a top view of an example hollow needle 110, being viewed from the tip 115 side.

FIG. 3D shows a partial side view of an example hollow needle 110.

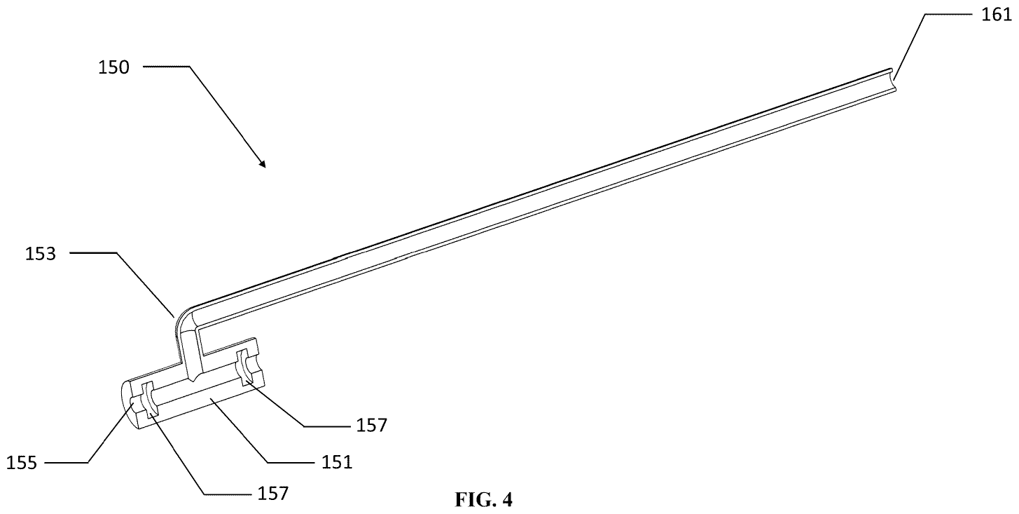

FIG. 4 shows a perspective view of an example needle holder 150 cut in half along an axis of the needle holder 150. The needle holder 150 may support the hollow needle 110 rotatably and connect the suction void 121 and the suction device fluidly. In some embodiments, the needle holder 150 may be coaxial with the tube axis 119 of the tube body 111 when the tube body 111 is supported by the needle holder 150.

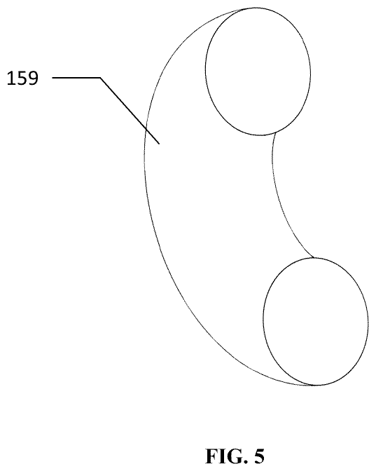

In some embodiments, the needle holder 150 comprises a needle holding portion 151 and a connector portion 153. The needle holding portion 151 may include a holder hollow 155 configured to receive the tube body 111. In various embodiments, the needle holder 150 comprises bearing supports 157 configured to hold bearings 159 (see FIG. 5). In some embodiments, the bearing support 157 is configured as a recess in the needle holding portion 151. The tube body 111 supported by the needle holder 150 may penetrate the needle holding portion 151 such that the tip 115 and the second end 117 extend from the needle holding portion 151.

In some embodiments, the connector portion 153 includes a portion that extends perpendicularly from the holding portion 151, which constitutes T-shape together with the needle holding portion 151. The connector portion 153 may include a connector 161 at an end. The connector 161 may be fluidly connected to the suction device. In various embodiments, the connector portion 153 may be curved in any appropriate direction.

FIG. 5 shows a perspective view of an example bearing 159 cut in half along an axis. In some embodiments, the bearing 159 is coaxial with the tube axis 119 of the tube body 111 when the tube body 111 is supported by the bearing 159. In some embodiments, the bearing 159 is configured to provide an airtight seal between needle holding portion 151 and the tube body 111 so that the suction force may be effectively transmitted to the hollow portion 113 of the tube body 111. For example, the bearing 159 comprises a ring made of elastic material that fits tightly against the needle holding portion 151 and the tube body 111 to ensure the airtight seal. In some embodiments, the suction void 121 of the hollow needle 110 may be located between the bearings 159 in the needle holding portion 151. In various embodiments, the bearing keeps the airtight seal even during the rotation of the tube body 111.

In some embodiments, the kit 100 includes a sensor (not shown) that detects the negative pressure in the hollow portion 113 caused by the suction device. The sensor may be connected anywhere in the fluid path from the suction device to the opening in the tip 115. In some embodiments, the kit 100 includes an indicator (not shown) that indicates a degree of the negative pressure detected by the sensor. In some embodiments, the suction device may comprise the sensor and the indicator. Alternatively, or additionally, the sensor and/or the indicator may be provided as separate devices from the suction device. In various embodiments, the indicator may be configured to show an increase of the negative pressure when the hollow needle 110 pulls the membrane by the negative pressure. The indicator may be configured to show a decrease of the negative pressure when the hollow needle 110 cut the membrane in a round shape and suck the round fragment of the membrane into the hollow portion 113.

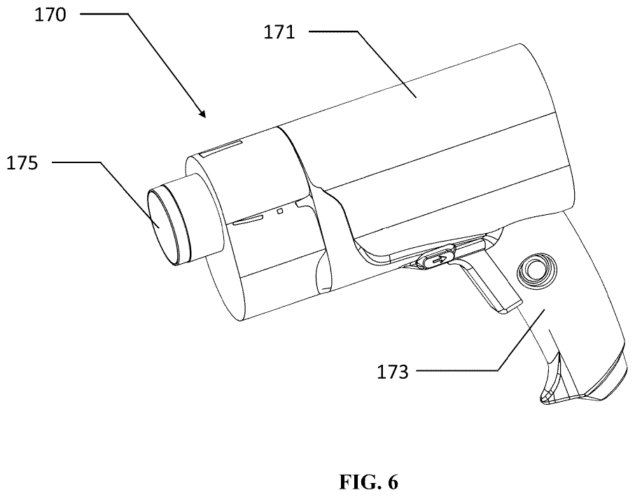

FIG. 6 shows a perspective view of an example rotating tool 170. The rotating tool may be a drill that may be electrically driven to cause torque. In some embodiments, the rotating tool 170 includes a drive body 171 and a handle 173. In various embodiments, drive body 171 comprises a chuck 175 configured to connect to an engagement shaft located at the second end 117 of the tube body 111. The rotating tool 170 may be configured to transmit torque to the hollow needle 110 through the connection provided by the chuck 175.

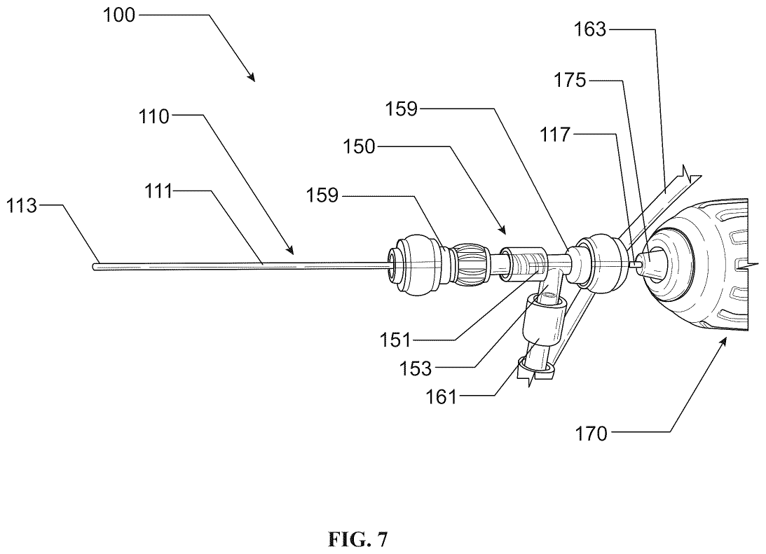

FIG. 7 shows a perspective view of an example kit 100. In some embodiments, the hollow needle 110 is rotatably supported by the bearings 159 of the needle holder 150. In some examples, the needle holder is configured as a T-shape. In some embodiments, a connector 161 is provided at the bottom of the needle holder in T-shape. In some embodiments, the connector 161 configured to connect to a first end of a hose 163, where a second end of the hose 163 may be connected to the suction device. In various embodiments, the chuck 175 of the rotating tool 170 holds the engagement shaft at the second end 117 of the hollow needle 110.

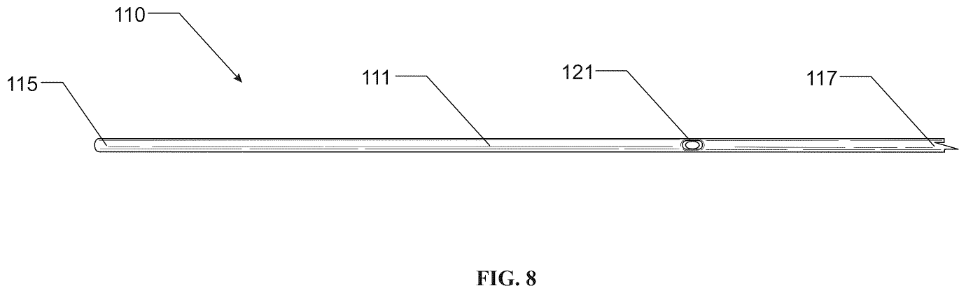

FIG. 8 shows a perspective view of an example hollow needle 110. As shown, the tube body 111 may include a suction void 121 between the tip 115 and the second end 117.

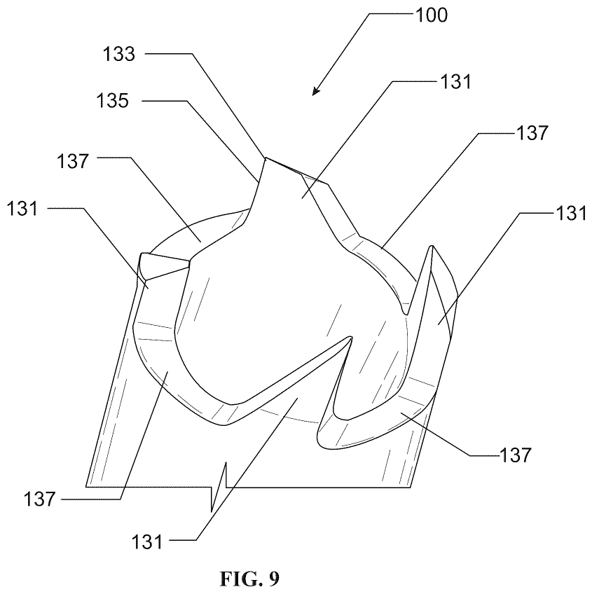

FIG. 9 shows a partial perspective view of an example hollow needle. In some embodiments, the side edge 135 of the sharp portion 131 may extend from the surface 137 substantially perpendicular to the surface 137.

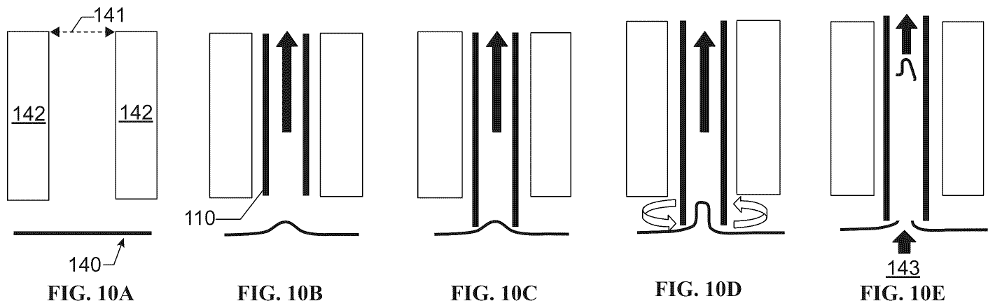

FIGS. 10A-10E shows schematic diagrams of an example hollow needle, which illustrate the flow of the use of the hollow needle 110 for making the void in the dura mater 140. In some examples, in an operation of a process to form a void in the dura mater 140, a burr hole is created in the skull 142 so that the dura mater 140 may be accessed through the burr hole (FIG. 10A). In some embodiments, a diameter 141 of the burr hole is 1 mm to 2 mm. For example, a 1 mm burr hole may be formed in the skull 142 to enable access to the dura mater 140.

In some examples, a negative pressure is applied at the tip 115 of the hollow needle 110 by the suction device, and the tip 115 is moved toward the dura mater 140 (FIG. 10B). In some embodiments, the diameter of the hollow needle 110 is 0.5 mm to 0.7 mm. For example, the diameter of the hollow needle 110 may be 0.6 mm. The hollow needle 110 may be lowered while applying the negative pressure (also referred to herein as a “suction force”). In some embodiments, a sensor may read the low level of the negative pressure while there is a leakage. The low negative pressure may indicate that the tip 115 of the hollow needle 110 has not contacted the surface of the dura mater 140. During the manipulation of the hollow needle, negative pressure by the suction device may be constantly applied. In some embodiments, the negative pressure value is from approximately 0.1 to 1.0 atm.

In some embodiments, by advancing the tip 115 within sufficient proximity of the dura mater 140, the suction force may be applied to the dura mater 140 through the tip 115 (FIG. 10C). In some embodiments, the suction force causes the sharp portion 131 to penetrate into the dura mater 140 by pulling the dura mater 140 to the tip 115. In various embodiments, the pulling action detaches the dura mater 140 from the brain tissues. The flat surface 137 of the tip 115 may contact the dura mater 140 and prevent further penetration of the sharp portion 131 through the dura matter. In doing so, the flat surface 137 may reduce a likelihood of the sharp portion 131 penetrating into subdural brain tissues.

In various embodiments, the contact of the hollow needle 110 and the dura mater 140 causes an increase in the negative pressure. For example, due to the tighter seal between the hollow needle 110 and the dura mater 140, the negative pressure at the tip 115 may increase. In some embodiments, a sensor may read the increase in negative pressure. In some embodiments, the increase in negative pressure is reported to the operator, where the increase may indicate to the operator the contact between the tip 115 and the dura mater 140. For example, in response to a sensor reading an increase level of the negative pressure, the operator may halt advancement of the hollow needle 110 to avoid damaging the subdural tissues.

In some embodiments, while the tip 115 is in contact with the dura mater 140, the hollow needle 110 is rotated to cut the membrane (FIG. 10D). The rotation of the hollow needle 110 may cause the side edge 135 of the sharp portion 131 to slice through the dura mater 140 in a circular manner. In doing so, the hollow needle 110 may form a round void extending through the dura mater 140. The rotating operation may be initiated subsequent to the piercing operation to ensure that the flat surface 137 stops the dura mater 140 in instances where the hollow needle 110 approaches the dura mater 140 unintentionally quickly.

In some embodiments, the rotation of the hollow needle 110 is halted following formation of the circular void. For example, the hollow needle 110 may be rotated by 360 degrees, 540 degrees, or another suitable value to ensure formation of the void via circular slicing of the dura mater 140. In some embodiments, as the dura mater 140 is cut, the constant negative pressure suctions cut material of the dura into the hollow portion 113. In doing so, a round void may be formed in the dura mater 140 in the desired size for to accommodate a designated sensor guidewire. In some embodiments, after formation of the void, the tip 115 may come out of contact with the dura mater 140. Additionally, or alternatively, following void formation the negative pressure in the hollow needle 110 may decrease (e.g., as the cut material, air, fluid, and/or the like traverse through the hollow portion 113). A sensor may read the decrease in pressure. The decrease in pressure may be reported to the operator, which may be interpreted as a signal of completion of the cutting of the dura mater 140. For example, an operator may halt rotation of the hollow needle 110 in response to an indication of decreased pressure within the hollow portion 113, at the tip 115, and/or the like.

In some embodiments, the hollow needle 110 is removed from the burr hole of the skull 142 (FIG. 10E). The negative pressure may be maintained throughout removal of the hollow needle 110 to ensure removal of cut material from the target site. Alternatively, in some embodiments, the application of negative pressure is suspended (or the negative pressure is reduced) during removal of the hollow needle 110 from the target site. In some embodiments, in response to detection of a drop in the negative pressure within the hollow needle, the application of the negative pressure is suspended. For example, aspiration of cut material into the hollow needle 110 may result in a drop in the negative pressure. The application of negative pressure may be suspended or reduced in response to detection of the reduced pressure, which may reduce aspiration of fluid into the hollow needle 110. In various embodiments, following removal of the hollow needle 110, one or more instruments may be inserted through the burr hole 143 of the skull and advanced through the void of the dura to access subdural tissues. For example, a sensor guidewire for epilepsy diagnosis may be inserted through the skull and dura to enable generation of sensor readings from subdural tissues.

### CONCLUSION

While some embodiments described herein relate to hollow needles, one of ordinary skill in the art will appreciate that the teachings herein may also apply to a wide range of medical procedures and apparatuses. The embodiments described herein may also be scalable to accommodate at least the aforementioned applications. Various components of embodiments described herein may be added, removed, reorganized, modified, duplicated, or the like as one skilled in the art would find convenient and/or necessary to implement a particular application in conjunction with the teachings of the present disclosure. In some embodiments, specialized features, characteristics, materials, components, and/or equipment may be applied in conjunction with the teachings of the present disclosure as one skilled in the art would find convenient and/or necessary to implement a particular application.

Moreover, many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of any appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of any appended claims. In this regard, for example, different combinations of elements and/or functions than those explicitly described above are also contemplated as may be set forth in some of any appended claims. Although specific terms are employed herein, they are used in a generic and descriptive sense only and not for purposes of limitation.

That which is claimed is:

1. A hollow needle for making a void in a membrane, the hollow needle comprising:
a tube body that extends along a tube axis between a first end and a second end, wherein:
the first end defines a tip of the tube body;
the tip comprises a sharp portion; and
the second end defines a suction void configured to receive a negative pressurization from a suction device;
a hollow portion surrounded by the tube body, wherein the hollow portion extends through and defines an opening of the tip, wherein:
the hollow portion is configured to apply the negative pressure through the opening to suction a membrane to the tip; and
the sharp portion is configured to cut the membrane via rotation of the tube body.

2. The hollow needle of claim 1, wherein:
the tube body comprises multiple sharp portions that are in an annular arrangement at the tip.

3. The hollow needle of claim 1, wherein
the sharp portion comprises a protrusion comprising:
a sharp tip configured to pierce the membrane; and
a side edge configured to cut the membrane via rotation of the tube body.

4. The hollow needle of claim 3, wherein
the side edge is arranged substantially parallel to the tube axis.

5. The hollow needle of claim 1, wherein:
the tube body further comprises a flat surface at the tip that is arranged to be substantially perpendicular to the tube axis; and
the sharp portion protrudes from the flat surface.

6. The hollow needle of claim 5, wherein:
the sharp portion protrudes from the flat surface by 0.2 mm to 4.0 mm.

7. The hollow needle of claim 6, wherein:
the sharp portion protrudes from the flat surface by at least 0.3 mm.

8. The hollow needle of claim 1, wherein:
the hollow portion is closed in the second end; and
the suction void is provided at a circumferential portion of the tube body between the tip and the second end.

9. The hollow needle of claim 1, wherein:
the tube body further comprises an engagement shaft configured to connect to a rotating tool.

10. The hollow needle of claim 9, wherein:
the engagement shaft is provided at the second end.

11. The hollow needle of claim 9, wherein:
the engagement shaft comprises a solid, flat portion configured to contact and halt advancement of the tip into the membrane.

12. The hollow needle of claim 1, wherein:
a diameter of the tube body is 0.5 mm to 0.7 mm.

13. The hollow needle of claim 1, wherein:
the membrane is a dura mater that surrounds a brain.

14. A kit comprising the hollow needle of claim 1, wherein:
the kit further comprises a needle holder comprising:
a holder body comprising a connector configured to be fluidly connected to the suction device; and
a bearing configured to rotatably support the tube body, wherein the needle holder is configured to receive the suction void.

15. The kit of claim 14, wherein:
the bearing is further configured to provide an airtight seal between the holder body and the tube body.

16. The kit of claim 14, wherein:
the tube body further comprises an engagement shaft configured to connect to a rotating tool, wherein the engagement shaft is provided at the second end of the tube body;
the holder body comprises a needle holding portion and a connector portion;
the tube body extends into the needle holding portion such that the tip and the second end extend from the needle holding portion; and
the connector portion comprises the connector.

17. The kit of claim 14, wherein:
the kit further comprises the suction device that may be fluidly connected to the connector.

18. The kit of claim 14, wherein
the kit further comprises:
a sensor configured to measure the negative pressure; and
an indicator configured to report a degree of the negative pressure measured by the sensor.

19. The kit of claim 14 wherein:
the tube body further comprises an engagement shaft; and
the kit further comprises a rotating tool configured to hold the engagement shaft.

20. A method for making a void in a membrane using the hollow needle of claim 1, wherein the method comprises:
applying the negative pressure at the tip by the suction device; and
advancing the tip toward the membrane, wherein the negative pressure pulls a portion of the membrane to the tip such that the sharp portion pierces the portion of the membrane.

21. The method of claim 20, wherein the method further comprises:
halting advancement of the tip toward the membrane in response to detecting an increase in the negative pressure.

22. The method of claim 20, wherein the method further comprises:
rotating the tube body to cause the sharp portion to cut a void into the portion of the membrane.

23. The method of claim 22, wherein the method further comprises:
halting rotation of the tube body in response to detecting a decrease in the negative pressure.

24. The method of claim 20, wherein the method further comprises:
suctioning into the hollow portion a cut fragment of the portion of the membrane.
